(19) United States
(12) Patent Application Publication
(10) Pub. No.: US 2021/0005307 A1
(43) Pub. Date: Jan. 7, 2021

(54) IMAGE PROCESSING AND ROUTING USING AI ORCHESTRATION

(71) Applicant: GE Precision Healthcare LLC, Wauwatosa, WI (US)

(72) Inventors: Jerome Knoplioch, Buc (FR); Paulo Gallotti Rodrigues, San Ramon, CA (US); Huy-Nam Doan, San Ramon, CA (US)

(21) Appl. No.: 16/503,065

(22) Filed: Jul. 3, 2019

(51) Int. Cl. G16H 30/40; G16H 10/60; G16H 30/20; G06N 20/00

(52) U.S. Cl. CPC G16H 30/40; G06N 20/00; G16H 30/20; G16H 10/60

(57) ABSTRACT

Systems, methods, and apparatus to generate and utilize predictive workflow analytics and inferencing are disclosed and described. An example apparatus includes an algorithm orchestrator to analyze medical data and associated metadata and select an algorithm based on the analysis. The example apparatus includes a postprocessor to execute the algorithm with respect to the medical data using one or more processing elements. In the example apparatus, the one or more processing elements are to be dynamically selected and arranged in combination by the algorithm orchestrator to implement the algorithm for the medical data, the postprocessor to output a result of the algorithm for action by the algorithm orchestrator.

### FIELD OF THE DISCLOSURE

This disclosure relates generally to image processing and, more particularly, to image processing and routing using artificial intelligence orchestration.

### BACKGROUND

The statements in this section merely provide background information related to the disclosure and may not constitute prior art.

Healthcare entities such as hospitals, clinics, clinical groups, and/or device vendors (e.g., implants) often employ local information systems to store and manage patient information. If a first healthcare entity having a first local information system refers a patient to a second healthcare entity having a second local information system, personnel at the first healthcare entity typically manually retrieves patient information from the first information system and stores the patient information on a storage device such as a compact disk (CD). The personnel and/or the patient then transport the storage device to the second healthcare entity, which employs personnel to upload the patient information from the storage device onto the second information system.

Additionally, modern radiology involves normalized review of image sets, detection of possible lesions/abnormalities and production of new images. Current processing of images, however, is labor-intensive and slow. Consistency of review formats and analysis results is limited by operator availability, skills and variability. Further, a number of processing actions require access to expensive dedicated hardware, which is not easily or affordably obtained.

### BRIEF DESCRIPTION

Systems, methods, and apparatus to generate and utilize predictive workflow analytics and inferencing are disclosed and described.

Certain examples provide an apparatus including an algorithm orchestrator to analyze medical data and associated metadata and select an algorithm based on the analysis. The example apparatus includes a postprocessor to execute the algorithm with respect to the medical data using one or more processing elements. In the example apparatus, the one or more processing elements are to be dynamically selected and arranged in combination by the algorithm orchestrator to implement the algorithm for the medical data, the postprocessor to output a result of the algorithm for action by the algorithm orchestrator.

Certain examples provide a computer-readable storage medium including instructions. The instructions, when executed by at least one processor, cause the at least one processor to at least: analyze medical data and associated metadata of a medical study; select an algorithm based on the analysis; dynamically select, arrange, and configure processing elements in combination to implement the algorithm for the medical data; execute the algorithm with respect to the medical data using the arranged, configured processing elements; and output an actionable result of the algorithm for the medical study.

Certain examples provide a computer-implemented method including: analyzing, by executing an instruction with at least one processor, medical data and associated metadata of a medical study; selecting, by executing an instruction with the at least one processor, an algorithm based on the analysis; dynamically selecting, arranging, and configuring, by executing an instruction with the at least one processor, processing elements in combination to implement the algorithm for the medical data; executing, by executing an instruction with the at least one processor, the algorithm with respect to the medical data using the arranged, configured processing elements; and outputting, by executing an instruction with the at least one processor, an actionable result of the algorithm for the medical study.

### BRIEF DESCRIPTION OF DRAWINGS

FIG. 1 is an example cloud-based clinical information system.

FIG. 2 illustrates an example imaging workflow processor that can be implemented in a system such as the example cloud-based clinical information system of FIG. 1.

FIG. 3 illustrates an example architecture to implement the imaging workflow processor of FIG. 2.

FIG. 4 illustrates an example of algorithm orchestration and inferencing services to execute in conjunction with the algorithm orchestrator of FIGS. 2-3.

FIG. 5 shows an example algorithm orchestration process to dynamically process study data using the algorithm orchestrator of FIGS. 2-4.

FIG. 6 depicts an example data flow to orchestrate workflow execution using the algorithm orchestrator of FIGS. 2-4.

FIGS. 7-8 illustrate flow diagrams of example methods to process a medical study using the example system(s) of FIGS. 2-4.

FIGS. 9-11 illustrate example algorithms dynamically constructed by the example systems of FIGS. 2-4 from a plurality of node models.

FIG. 12 illustrates a flow diagram of an example algorithm orchestration process to augment clinical workflows using the algorithm orchestrator of FIGS. 2-4.

FIG. 13 depicts an example chest x-ray workflow for pneumothorax detection that can be assembled and executed via the algorithm orchestrator of FIGS. 2-4.

FIG. 14 is a block diagram of an example processor platform capable of executing instructions to implement the example systems and methods disclosed and described herein.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object.

As used herein, the terms “system,” “unit,” “module,” “engine,” etc., may include a hardware and/or software system that operates to perform one or more functions. For example, a module, unit, or system may include a computer processor, controller, and/or other logic-based device that performs operations based on instructions stored on a tangible and non-transitory computer readable storage medium, such as a computer memory. Alternatively, a module, unit, engine, or system may include a hard-wired device that performs operations based on hard-wired logic of the device. Various modules, units, engines, and/or systems shown in the attached figures may represent the hardware that operates based on software or hardwired instructions, the software that directs hardware to perform the operations, or a combination thereof.

As used herein, singular references (e.g., “a”, “an”, “first”, “second”, etc.) do not exclude a plurality. The term “a” or “an” entity, as used herein, refers to one or more of that entity. The terms “a” (or “an”), “one or more”, and “at least one” can be used interchangeably herein. Furthermore, although individually listed, a plurality of means, elements or method actions may be implemented by, e.g., a single unit or processor. Additionally, although individual features may be included in different examples or claims, these may possibly be combined, and the inclusion in different examples or claims does not imply that a combination of features is not feasible and/or advantageous.

The term “and/or” when used, for example, in a form such as A, B, and/or C refers to any combination or subset of A, B, C such as (1) A alone, (2) B alone, (3) C alone, (4) A with B, (5) A with C, (6) B with C, and (7) A with B and with C. As used herein in the context of describing structures, components, items, objects, and/or things, the phrase “at least one of A and B” is intended to refer to implementations including any of (1) at least one A, (2) at least one B, and (3) at least one A and at least one B. Similarly, as used herein in the context of describing structures, components, items, objects and/or things, the phrase “at least one of A or B” is intended to refer to implementations including any of (1) at least one A, (2) at least one B, and (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, and (3) at least one A and at least one B. Similarly, as used herein in the context of describing the performance or execution of processes, instructions, actions, activities, and/or steps, the phrase “at least one of A or B” is intended to refer to implementations including any of (1) at least one A, (2) at least one B, and (3) at least one A and at least one B.

In addition, it should be understood that references to “one embodiment” or “an embodiment” of the present disclosure are not intended to be interpreted as excluding the existence of additional embodiments that also incorporate the recited features.

Aspects disclosed and described herein provide systems and associated methods to process and route image and related healthcare data using artificial intelligence (AI) orchestration.

An example cloud-based clinical information system described herein enables healthcare entities (e.g., patients, clinicians, sites, groups, communities, and/or other entities) to share information via web-based applications, cloud storage and cloud services. For example, the cloud-based clinical information system may enable a first clinician to securely upload information into the cloud-based clinical information system to allow a second clinician to view and/or download the information via a web application. Thus, for example, the first clinician may upload an x-ray image into the cloud-based clinical information system (and/or the medical image can be automatically uploaded from an imaging system to the cloud-based clinical information system), and the second clinician may view the x-ray image via a web browser and/or download the x-ray image onto a local information system employed by the second clinician.

In some examples, a first healthcare entity may register with the cloud-based clinical information system to acquire credentials and/or access the cloud-based clinical information system. To share information with a second healthcare entity and/or gain other enrollment privileges (e.g., access to local information systems), the first healthcare entity enrolls with the second healthcare entity. In some examples, the example cloud-based clinical information system segregates registration from enrollment. For example, a clinician may be registered with the cloud-based clinical information system and enrolled with a first hospital and a second hospital. If the clinician no longer chooses to be enrolled with the second hospital, enrollment of the clinician with the second hospital can be removed or revoked without the clinician losing access to the cloud-based clinical information system and/or enrollment privileges established between the clinician and the first hospital.

In some examples, business agreements between healthcare entities are initiated and/or managed via the cloud-based clinical information system. For example, if the first healthcare entity is unaffiliated with the second healthcare entity (e.g., no legal or business agreement exists between the first healthcare entity and the second healthcare entity) when the first healthcare entity enrolls with the second healthcare entity, the cloud-based clinical information system provides the first healthcare entity with a business agreement and/or terms of use that the first healthcare entity executes prior to being enrolled with the second healthcare entity. The business agreement and/or the terms of use may be generated by the second healthcare entity and stored in the cloud-based clinical information system. In some examples, based on the agreement and/or the terms of use, the cloud-based clinical information system generates rules that govern what information the first healthcare entity may access from the second healthcare entity and/or how information from the second healthcare entity may be shared by the first healthcare entity with other entities and/or other rules.

In some examples, the cloud-based clinical information system may employ a hierarchal organizational scheme based on entity types to facilitate referral network growth, business agreement management, and regulatory and privacy compliance. Example entity types include patients, clinicians, groups, sites, integrated delivery networks, communities and/or other entity types. A user, which may be a healthcare entity or an administrator of a healthcare entity, may register as a given entity type within the hierarchal organizational scheme to be provided with predetermined rights and/or restrictions related to sending information and/or receiving information via the cloud-based clinical information system. For example, a user registered as a patient may receive or share any patient information of the user while being prevented from accessing any other patients' information. In some examples, a user may be registered as two types of healthcare entities. For example, a healthcare professional may be registered as a patient and a clinician.

In some examples, the cloud-based clinical information system includes an edge device located at healthcare facility (e.g., a hospital). The edge device may communicate with a protocol employed by the local information system(s) to function as a gateway or mediator between the local information system(s) and the cloud-based clinical information system. In some examples, the edge device is used to automatically generate patient and/or exam records in the local information system(s) and attach patient information to the patient and/or exam records when patient information is sent to a healthcare entity associated with the healthcare facility via the cloud-based clinical information system.

In some examples, the cloud-based clinical information system generates user interfaces that enable users to interact with the cloud-based clinical information system and/or communicate with other users employing the cloud-based clinical information system. An example user interface described herein enables a user to generate messages, receive messages, create cases (e.g., patient image studies, orders, etc.), share information, receive information, view information, and/or perform other actions via the cloud-based clinical information system.

In certain examples, images are automatically sent to a cloud-based information system. The images are processed automatically via “the cloud” based on one or more rules. After processing, the images are routed to one or more of a set of target systems.

Routing and processing rules can involve elements included in the data or an anatomy recognition module which determines algorithms to be applied and destinations for the processed contents. The anatomy module may determine anatomical sub-regions so that routing and processing is selectively applied inside larger data sets. Processing rules can define a set of algorithms to be executed on an input data set, for example. Modern radiology involves normalized review of image sets, detection of possible lesions/abnormalities and production of new images (functional maps, processed images) and quantitative results. Some examples of very frequent processing include producing new slices along specific anatomical conventions to better highlight anatomy (e.g., discs between vertebrae, radial reformation of knees, many musculo-skeletal views, etc.). Additionally, processing can be used to generate new functional maps (e.g., perfusion, diffusion, etc.), as well as quantification of lesions, organ sizes, etc. Automated identification of vascular system can also be processed.

In contrast to labor-intensive, slow, inconsistent traditional processing, a leveraging of cloud resources would open access to large amounts of compute resources and enable automated production of intermediate or final results (new images, quantitative results). It is, however, very difficult to launch the right algorithms automatically. Traditional systems try to guess anatomy and intention of scan from additional information in an image header. Such guesswork is usually very error prone, site dependent and not possible in situations where there is time pressure during scan (trauma, for example). This problem of guesswork also impacts productivity in interactive usages on analysis workstations, Picture Archiving and Communication Systems (PACS), and scanner consoles.

Additionally, high end cloud hardware is expensive to rent, but accessing a larger number of smaller nodes is cost effective compared to owning dedicated, on-premises hardware. Dispatching multiple tasks to a large number of small processing units allows more cost-effective operation, for example.

Although cloud storage can be an efficient model for long term handling of data, in medical cases, data sets are large and interactive performance from cloud-based rendering may not be guaranteed under all network conditions. Certain examples desirably push data sets automatically to one or more target systems. Intelligently pushing data sets to one or more target systems also avoids maintaining multiple medical image databases (e.g., Cloud storage may not be an option for sites that prefer their own vendor neutral archive (VNA) or PACS, etc.).

In certain examples, a user is notified when image content is available for routing. In other examples, a user is notified when processing has been performed and results are available. Thus, certain examples provide increases user productivity. For example, results are automatically presented to users, reducing labor time. Additionally, users can be notified when new data is available. Further, large data can be pushed to one or more local systems for faster review, saving networking time. An efficient selection of relevant views also helps provide a focused review and diagnostic, for example. Anatomy recognition results can be used to improve selection of appropriate hanging protocol(s) and/or tools in a final PACS or workstation reading, for example.

Certain examples improve quality and consistency of results through automation. Automated generation of results helps ensure that results are always available to a clinician and/or other user. Routing helps ensures that results are dispatched to proper experts and users. Cloud operation enables access across sites, thus reaching specialists no matter where they are located.

Certain examples also reduce cost of ownership and/or operation. For example, usage of Cloud resources versus local hardware should limit costs. Additionally, dispatching analysis to multiple nodes also reduces cost and resource stress on any particular node.

In certain examples, after pushing an image study, the study is forwarded to a health cloud. Digital Imaging and Communications in Medicine (DICOM) tags associated with the study are evaluated against one or more criteria, which trigger a corresponding algorithm. The image study can be evaluated according to anatomy detection, feature vector, etc. The algorithm output is then stored with the study. Additionally, a notification (e.g., a short message service (SMS) message, etc.) is sent upon algorithm completion, and results of the algorithm are pushed back to the original study. The study can be marked according to priority in a worklist depending on the algorithm output, for example. Study data can be processed progressively (e.g., streaming as the data is received) and/or once all the study is received, for example.

In certain examples, an orchestration layer can be used to configure instructions and define a particular sequence of processors and routers to process content (e.g., non-image data, image data of different types, etc.). The orchestration layer can configure processor(s) and/or router(s) to process and/or route according to certain criteria such as anatomy, etc. The orchestration layer can chain processors to arrange multiple processors in a sequence (e.g., lung segmentation followed by nodule identification, etc.), for example.

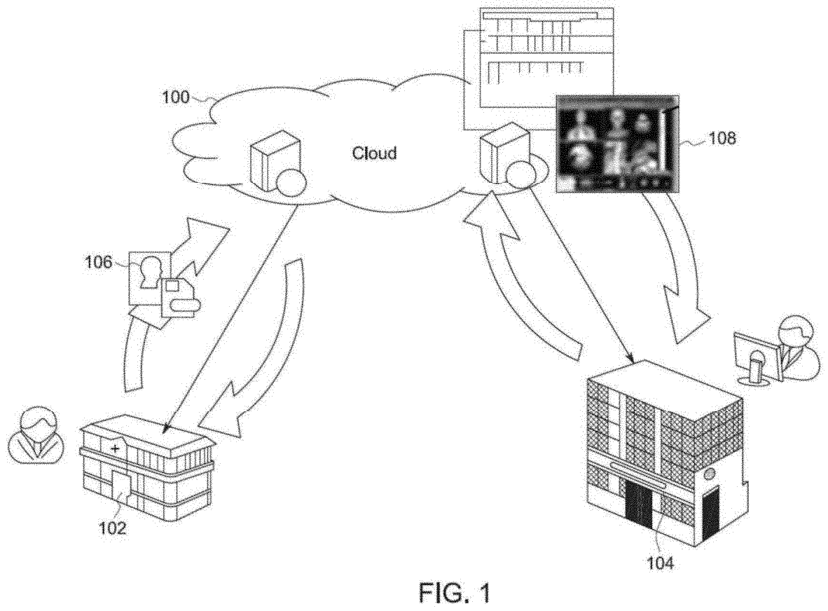

FIG. 1 illustrates an example cloud-based clinical information system 100 disclosed herein. In the illustrated example, the cloud-based clinical information system 100 is employed by a first healthcare entity 102 and a second healthcare entity 104. As described in greater detail below, example entity types include a community, an integrated delivery network (IDN), a site, a group, a clinician, and a patient and/or other entities.

In the illustrated example, the first healthcare entity 102 employs the example cloud-based clinical information system 100 to facilitate a patient referral. Although the following example is described in conjunction with a patient referral (e.g., a trauma transfer), the cloud-based information system 100 may be used to share information to acquire a second opinion, conduct a medical analysis (e.g., a specialist located in a first location may review and analyze a medical image captured at a second location), facilitate care of a patient that is treated in a plurality of medical facilities, and/or in other situations and/or for other purposes.

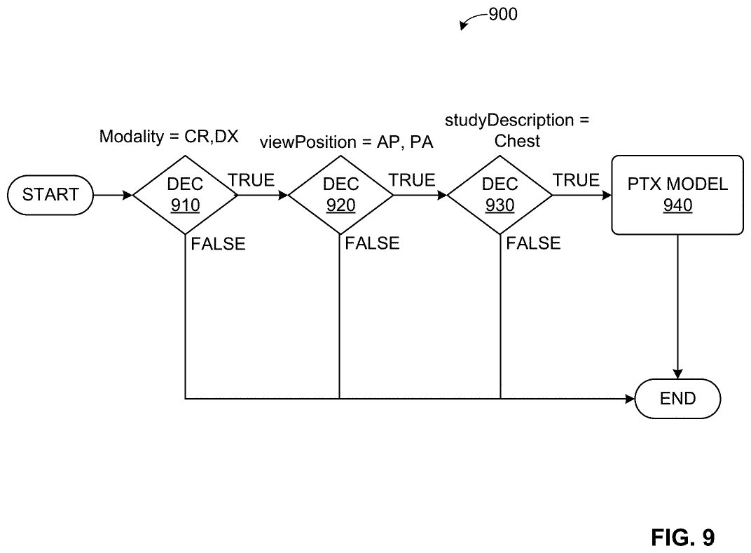

In the illustrated example of FIG. 1, the first healthcare entity 102 may be a medical clinic that provides care to a patient. The first healthcare entity 102 generates patient information (e.g., contact information, medical reports, medical images, and/or any other type of patient information) associated with the patient and stores the patient information in a first local information system (e.g., PACS/RIS and/or any other local information system). To refer the patient to the second healthcare entity 104, the first healthcare entity posts or uploads an order 106, which includes relevant portions of the patient information, to the cloud-based clinical information system 100 and specifies that the patient is to be referred to the second healthcare entity. For example, the first healthcare entity 102 may use a user interface (FIGS. 9-11) generated via the cloud-based clinical information system 100 to upload the order 106 via the internet from the first local information system to the cloud-based clinical information system 100 and direct the cloud-based information system 100 notify the second healthcare entity 104 of the referral and/or enable the second healthcare entity 104 to access the order 106. In some examples, the cloud-based clinical information system 100 generates a message including a secure link to the order 106 and emails the message to the second healthcare entity 104. The second healthcare entity 104 may then view the order 106 through a web browser 108 via the cloud-based clinical information system 100, accept and/or reject the referral, and/or download the order 106 including the patient information into a second local information system (e.g., PACS/RIS) of the second healthcare entity 104. As described in greater detail below, the cloud-based-based clinical information system 100 manages business agreements between healthcare entities to enable unaffiliated healthcare entities to share information, thereby facilitating referral network growth.

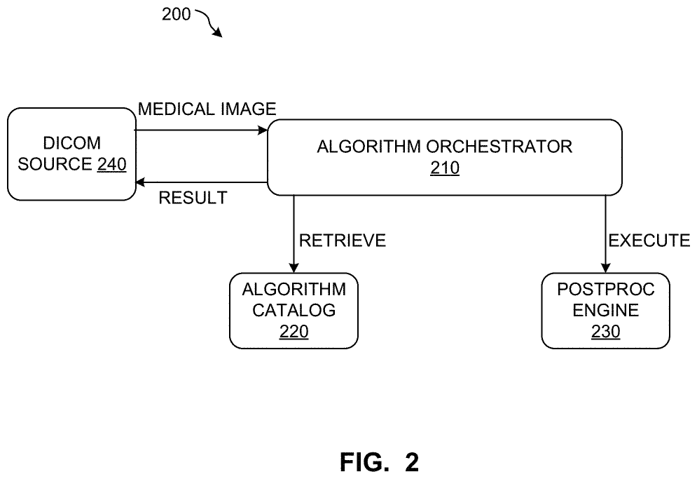

FIG. 2 illustrates an example imaging workflow processor 200 that can be implemented in a system such as the example cloud-based clinical information system 100 of FIG. 1. The example imaging workflow processor 200 can be a separate system and/or can be implemented in a PACS, RIS, vendor-neutral archive (VNA), an image viewer, etc., to connect such systems with algorithms created by different providers to process image data.

The example imaging workflow processor 200 includes an algorithm orchestrator 210, an algorithm catalog 220, and a postprocessing engine 230 interacting with a DICOM source 240 to obtain medical image(s). As shown in the example of FIG. 2, the DICOM source 240 provides a medical image to the algorithm orchestrator 210, which identifies and retrieves a corresponding algorithm for that image from the algorithm catalog 220 and executes the algorithm using the postprocessing engine 230. A result of the algorithm execution with respect to the medical image is output and provided back to the DICOM source 240, for example. As such, given a medical image, the algorithm orchestrator 210 facilitates a workflow of postprocessing based on a catalog 220 of algorithms compatible with that image to produce consumable outcomes.

In certain examples, a medical image is defined as an output of an imaging modality (e.g., x-ray, computed tomography (CT), magnetic resonance (MR), ultrasound, etc.) stored as one or more DICOM files in the DICOM Source or repository 240. A DICOM file includes metadata with patient, study, series, and image information as well as image pixel data, for example. A workflow includes an orchestrated and repeatable pattern of services calls to process DICOM study information, execute algorithms, and produce outcomes to be consumed by other systems, for example. In this context, postprocessing can be defined as a sequence of algorithms executed after the image has been acquired from the modality to enhance the image, transform the image, and/or extract information that can be used to assist a radiologist to diagnose and treat a disease, for example. An algorithm is a sequence of computational processing actions used to transform an input image into an output image with a particular purpose or function (e.g., for computer-aided detection, for radiology reading, for automated processing, for comparison, etc.).

In certain examples, five classes of algorithms can be used in image postprocessing: image restoration, image analysis, image synthesis, image enhancement, and image compression. Image restoration is used to improve the quality of the image. Image analysis is applied to identify condition(s) (in a classification model) and/or region(s) of interest (in a segmentation model) in an image. Image synthesis is used to construct a three-dimensional (3D) image based on multiple two-dimensional (2D) images. Image enhancement is applied to improve the image by using filters and/or adding information to assist with visualization. Image compression is to reduce the size of the image to enhance transmission times and storage involved in storing the image, for example. Algorithms can be implemented using one or more machine learning and/or deep learning models, other artificial intelligence, and/or other processing to apply the algorithm(s) to the image(s), for example. Outcomes are artifacts produced by an algorithm executed using one or more medical images as input. The outcomes can be in different formats, such as: DICOM structured report (SR), DICOM secondary capture, DICOM parametric map, image, text, JavaScript Object Notation (JSON), etc.

In certain examples, the algorithm orchestrator 210 interacts with one or more types of systems including an imaging provider (e.g., a DICOM modality also known as a DICOM source 240, a PACS, a VNA, etc.), a viewer (e.g., a DICOM viewer that displays the results of the algorithms executed by the orchestrator 210, etc.), the algorithm catalog 220 (e.g., a repository of algorithms available for different types of imaging modalities, etc.), an inferencing engine (e.g., a system or component such as the postprocessing engine 230 that is able to run an algorithm based on input parameters and produce an output, etc.), other system (e.g., one or more external entities that receive notifications from an orchestration workflow (e.g., a RIS, etc.), etc.).

The algorithm orchestrator 210 can be used by one or more applications to execute algorithms on medical images according to pre-defined workflows, for example. An example workflow includes actions formed from a plurality of action types including: Start, End, Decision, Task, Model and Wait. Start and End actions define where the workflow starts and ends. A Decision action is used to evaluate expressions to define the next action to be executed (similar to a switch-case instruction in programming languages, for example). A Task action represents a synchronous call to a REST service. A Model action is used to execute an algorithm from the catalog 220. Wait tasks can be used to track the execution of asynchronous tasks as part of the orchestration and are used in operations that are time-consuming such as moving a DICOM study from a PACS to the algorithm orchestrator 210, pushing the algorithm results to the PACS, executing a deep learning model, etc. Workflows can aggregate the outcomes of different algorithms executed and notify other systems about the status of the orchestration, for example.

In example operation, a new image study can be provided from a PACS system (e.g., a cloud-based PACS system 100, etc.) to be processed by the orchestrator 210. For example, a hypertext transfer protocol (HTTP) request to a representational state transfer (REST) application programming interface (API) exposed by an API gateway called “study process notification” includes the imaging study metadata in the payload. The gateway forwards the request to the appropriate orchestration service that validates the request payload and responds with an execution identifier (ID) and a status. The orchestration service invokes available workflow(s) in the orchestration engine 210. Each workflow can be executed as a separate thread. A workflow may begin by validating DICOM metadata to determine whether the metadata matches workflow requirements (e.g., modality, view position, study description, etc.) and, in case of a match, transfers the study data from the PACS to a local file storage. When the transfer is complete, the orchestration engine 210 executes one or more algorithms defined in the workflow. For each algorithm that has to be executed, the orchestrator 210 invokes analytics as a service (AAAS) to execute the algorithm and awaits a response. Once the algorithm response(s) are available, the orchestrator 210 transfers resulting output file(s) produced by the algorithm(s) to the information system 100 (e.g., PACS, RIS, VNA, etc.) and sends a notification message saying the processing of that study is complete. The notification message also includes a list of algorithm(s) executed by the orchestrator 210 and the execution results for each algorithm, for example.

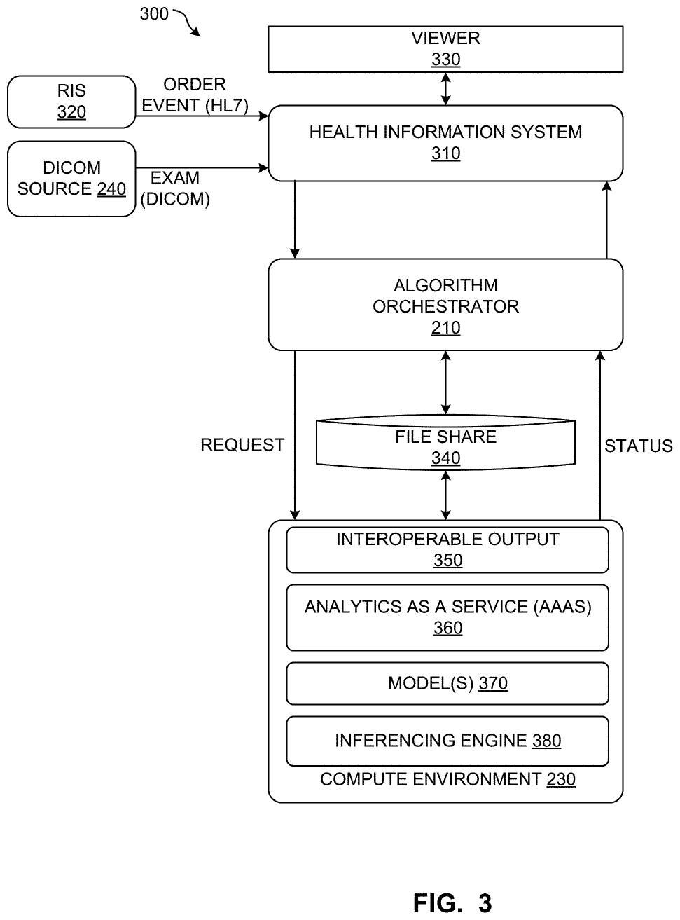

The example imaging workflow processor 200 can be viewed differently as shown in the example architecture 300 of FIG. 3. As shown in the example of FIG. 3, the DICOM source 240 communicates with a health information system 310, such as a PACS, EMR, enterprise archive (EA) (e.g., a VNA, etc.), fusion/combination system, etc., as well as a RIS 320, such that the RIS 320 provides an order event (e.g., an HL7 order event, etc.), and the DICOM source 240 provides exam data (e.g., DICOM data for an imaging exam, etc.) to the information system 310. The example information system 310 provides the exam data to the algorithm orchestrator 210. The example healthcare information system 310 also interacts with a viewer (e.g., a workflow manager, universal viewer, zero footprint viewer, etc.) to display an output/outcome of the selected algorithmic processing of the exam data from the algorithm orchestrator 210, etc. A file share 340 stores exam data from the algorithm orchestrator 210, processing results from the processor 230, etc.

As shown in the example of FIG. 3, the postprocessor and/or other computing environment 230 processes the exam data according to one or more determined algorithm(s) and associated information. The example computing environment 230 includes an interoperable output 350 providing algorithm(s), processing result(s), etc., to and from the computing environment 230, the file share 240, and the algorithm orchestrator 210. The example computing environment 230 also includes analytics as a service (AAAS) 360 to provide analytics to process the exam data, associated algorithm(s), resulting image(s), etc. In certain examples, the AAAS 360 provides the algorithm catalog 220 and associated algorithm registry from which algorithms are extracted to process the exam data. The example computing environment 230 includes one or more artificial intelligence (AI) models 370 and an inferencing engine 380 to generate and/or leverage the model(s) 370 with respect to the exam data and algorithm orchestrator 210, for example. The inferencing engine 380 can leverage the model(s) to apply one or more algorithms selected from the AAAS 360 algorithm catalog 220 to the exam data from the algorithm orchestrator 210, for example. The inferencing engine 380 takes the exam data, algorithm(s), and one or more input parameters and produces an output from processing the exam data (e.g., image restoration, etc.), which provided to the file share 340, algorithm orchestrator 210, and information system 310, for example. The output can be displayed for interaction via the viewer 330, for example.

In operation, for example, the algorithm orchestrator 210 can receive an exam and/or other data to be processed (e.g., image data, etc.) and connect that exam and associated healthcare information system 310 to a computing system/engine/environment 230 including algorithms created by different providers to apply different operations to image and/or other exam data to produce a displayable, interactable, and/or otherwise actionable output for the viewer 330, information system 310, etc. Exam data can be provided by the system 310 independently or in conjunction with the DICOM source 240 such as an imaging scanner, a workstation, etc. Based on characteristics of the exam data, the orchestrator 210 can select one or more algorithms from the AAAS 360 for processing. The inferencing engine 380 of the postprocessor 230 executes the algorithm(s) with respect to the exam data using one or more models 370, for example.

In certain examples, a plurality of models 370 and a plurality of algorithms can be allocated such that a plurality of physical and/or virtual machine processors can be instantiated to implement algorithms according to a series of rules, criteria, equations, network models, etc. For example, the orchestration engine 210 can first select a lung segmentation algorithm from the AAAS 360 to segment lung image data and then select a nodule identification algorithm from the AAAS 360 to identify nodules in the segmented lung image data. The algorithm orchestrator 210 can connect or chain algorithms, customize algorithm(s), and/or otherwise configure algorithms and define algorithm orchestration workflows to fit particular exam data, reason for exam, viewer 330 type, viewer 330 role, viewer 330 context, DICOM header information and/or other metadata (e.g., modality, series, study description, etc.), etc. In certain examples, a configured algorithm, workflow, etc., can be saved and stored in the file share 340 for later use by the information system 310, the viewer 330, etc.

In certain examples, the algorithm orchestrator 210 can handle a plurality of image and/or other exam data processing requests from a plurality of health information systems 310 and/or DICOM sources 240 using the computing infrastructure 230. In some examples, each request triggers the algorithm orchestrator 210 to spawn a virtual machine, Docker container, etc., to instantiate the respective algorithm from the AAAS 360 and any associated model(s) 370. A virtual machine, container, etc., can be instantiated to chain and/or otherwise combine results from other virtual machine(s), container(s), etc.,

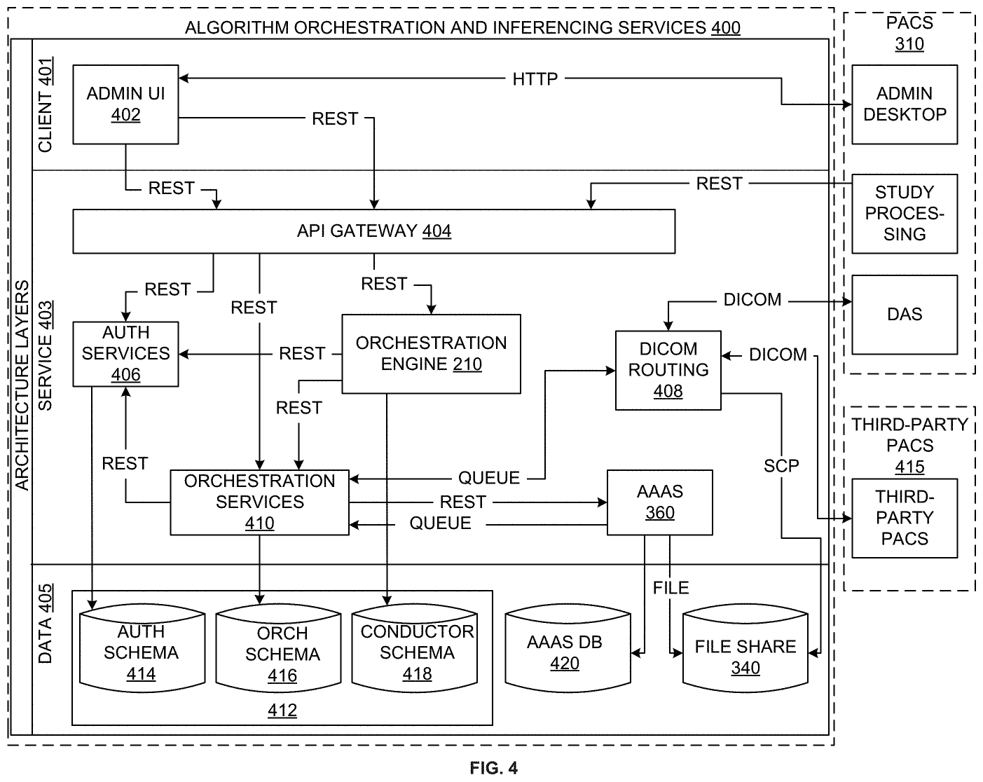

FIG. 4 illustrates an example of algorithm orchestration and inferencing services 400 to run in conjunction with the algorithm orchestrator 210. The example services 300 are implemented using a client layer 401, a service layer 403, and a data layer 405. The example client layer 401 includes an administrative user interface (UI) 402 to enable a user at an external system, such as the health information system 310 (illustrated in the example of FIG. 4 as a PACS but also applicable to other systems 310 such as RIS, EA, EMR, etc.), to interact with the algorithm orchestrator 210 to process and route image and/or other exam data (e.g., via HTTP, REST, DICOM, etc.). The example service layer 403 includes an API gateway 404 to route requests from the client layer 401 (e.g., via the UI 402). The example service layer 403 also includes authentication services 406, the orchestration engine 210, a DICOM router 408, orchestration services 410, and an AAAS 370. Elements of the service layer 403, such as the DICOM router 408, etc., can interact with another PACS 415, for example. The example data layer 405 includes a data store 412 including authorization schema 414, orchestration schema 416, conductor schema 418, etc. The data layer 305 of the example of FIG. 4 also includes an AAAS database 420 and a file share 350, for example.

Using the example architecture 400, the orchestration engine 210 can leverage the orchestration services 410 and the AAAS 360 to dynamically generate a workflow from models associated with processing algorithms in the AAAS database 420 and/or the file share 340, for example. For example, a pneumothorax (PTX) model 370 can be retrieved from the AAAS database 420 and provided by the AAAS 360 to the orchestration services 410 of the orchestration engine 210 to process image and/or other exam data to identify presence and/or likelihood of a pneumothorax. The PTX model is combined with a particular modality(-ies) (e.g., computed radiography (CR), digital x-ray (DX), etc.), view position (e.g., anteroposterior (AP), posteroanterior (PA), etc.), study description (e.g., chest, lung, etc.), etc., to form a processing workflow to which exam data can be applied, for example. In other examples, a fork can be introduced by the algorithm orchestrator 210 to determine whether the PTX model or an endotracheal (ET) tube model is to be applied to the data. In such an example, processing from both the PTX model and the ET tube model can proceed in parallel and be joined or combined to generate an output result. In another example, model processing is serial, such as first applying a position model and then applying the PTX model, etc.

In certain examples, workflows can be dynamically constructed by the algorithm orchestrator 210 using an extensible format to support a variety of tasks, workflows, etc. One or more nodes can dynamically be connected together, allocating processing, memory, and communication resources to instantiate a workflow. For example, a start node defines a beginning of a workflow. An end node defines an end of the workflow. A sub-workflow node invokes a sub-workflow that is also registered in the orchestration engine 210. An HTTP task node invokes an HTTP service using a method such as a POST, GET, PUT, PATCH, DELETE, etc. A wait task node is to wait for an asynchronous task to be completed. A decision node makes a flow decision based on a JavaScript expression, etc. A join node waits for parallel executions triggered by a fork node to be completed before proceeding, for example.

In an example, the PACS 310 has a new study to be processed through the orchestration engine 210. The PACS 310 sends an HTTP request to a REST API exposed by the API Gateway 404 referred to as a “study process notification” including the study metadata in the payload. The gateway 404 forwards the request to a corresponding orchestration service 410. The orchestration service 410 validates the request payload and responds with an execution ID and a status. The orchestration service 410 invokes available workflow(s) in the orchestration engine 210. Each workflow is executed as a separate thread. For example, a workflow can begin by validating associated DICOM metadata to determine whether the study's DICOM metadata matches workflow requirements (e.g., modality, view position, study description, etc.). When the metadata matches the workflow requirements, the orchestration engine 210 transfers the study data from the PACS 310 to local file storage 422. When the transfer is complete, the orchestration engine 210 executes algorithm(s) defined in the workflow. For each algorithm to be executed, the orchestration engine 210 invokes AAAS 360 and awaits a response. Once the response of all applicable algorithm(s) is available, the orchestration engine 210 transfers output file(s) produced by the algorithm(s) to the PACS 310. Once transferred, the orchestration engine 210 can send a notification message indicating that processing of that study is complete. This notification message can also include a list of algorithm(s) executed by the orchestration engine 210 with respect to the study and execution results for each algorithm.

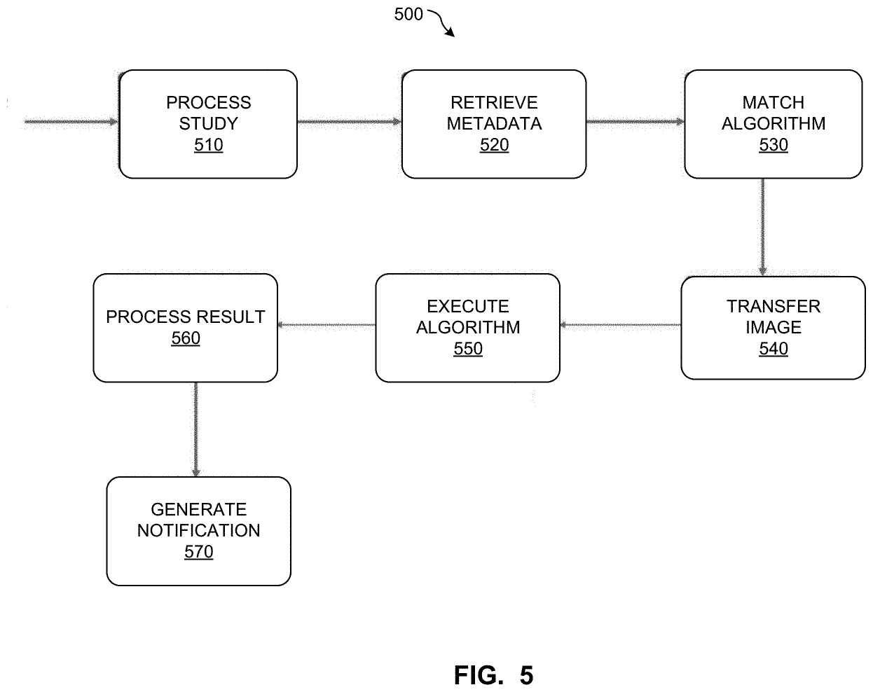

FIG. 5 shows an example algorithm orchestration process 500 to dynamically process study data using the algorithm orchestrator 210. At block 510, an input study is processed. For example, an imaging and/or other exam study is received via a gateway 404 upload, Web service upload, DICOM push, etc. The study is processed, such as by orchestration services 410, the orchestration engine 210, etc., to identify the study, etc. At block 520, metadata associated with the study is retrieved (e.g., from the file share 340, PACS 310, 415, etc.). For example, a RESTful service search query (e.g., QIDO-RS) can be executed, a C-FIND search command can be utilized, etc., to identify associated metadata.

At block 530, an algorithm is matched to the study by the algorithm orchestrator 210 based on the metadata. For example, a PTX identification algorithm is matched to the study based on the indication of lung images, air, etc., in the metadata. In certain examples, an algorithm is retrieved from storage (e.g., the AAAS database 420, the file share 340, etc.). In certain examples, an algorithm is dynamically constructed by the algorithm orchestrator 210 from elements (e.g., algorithms, nodes, functional code blocks, etc.) retrieved from storage (e.g., the AAAS database 420, the file share 340, etc.). At block 540, image data from the study is transferred (e.g., from the PACS 310 to the file share 340, other local file storage, etc.) such as using a C-MOVE server message block (SMB) shared file access, streaming, etc., so that the study data can be processed according to the example algorithm orchestration and inferencing services 400. At block 550, the matched algorithm is executed with respect to the transferred image data. For example, the AAAS 360 deploys one or more models 370 and/or other machine learning constructs to implement the algorithm and apply it to the image data. Tasks in the algorithm execution can proceed serially and/or in parallel on the image data, for example. In certain examples, some tasks may wait for other tasks to be completed and/or other information to be generated and/or otherwise become available, etc.

At block 560, result(s) of the algorithm are processed. For example, a probability, indication, detection, score, location, severity, and/or other prediction, conclusion, measure, etc., provided by the algorithm is processed (e.g., by the orchestration engine 210, inferencing engine 380 and/or other postprocessor 230 (e.g., provided by the AAAS 360 and/or orchestrator 210, etc.), etc.) to provide an actionable output, draw a conclusion, combine multiple algorithm results, etc. Result(s) can be stored in the file share 340, AAAS database 420, other data store, etc., using a command such as C-STORE, SMB shared access, etc. At block 570, a notification is generated. For example, results of image study processing can be displayed via the viewer 330, transmitted to the PACS and/or other information system 310, 415, etc., reported to the RIS 320 and/or DICOM source 240, etc., such as via REST Web service, HL7 message, SMS message, email, HTTP command, etc.

Thus, the example orchestrator 210 can provide a central engine to coordinate interaction between different services. The orchestrator 210 knows how to invoke each service an manage dependencies and transactions between services (e.g., in the orchestration services 410, AAAS 360, etc.). Alternatively or in addition, services can be choreographed to know which other service(s) to interact with in a distributed manner. In certain examples, the algorithm orchestrator 210 can support a plurality of different workflows based on the same set of services arranged in different compositions. A workflow is designed around the centralized orchestrator 210 and the same services 360, 410, etc., can be executed in different arrangements depending on the use case, for example.

In certain examples, the algorithm orchestrator 210 can facilitate algorithm onboarding/creation, update, and removal using the orchestration services 410 and the AAAS 360 to create an algorithm (e.g., potentially with input from an external source via the admin UI 402, etc.), list the algorithm, and save the algorithm via the orchestration schema database 416. In certain examples, the algorithm orchestrator 210 can facilitate workflow creation, activation, update, and removal using the orchestration services 410 to register a workflow and its associated tasks (e.g., potentially with input from an external source via the admin UI 402, etc.) and save the workflow via the orchestration schema database 416. When the algorithm orchestrator 210 receives a request (e.g., from the PACS and/or other information system 310, etc.) for a new study to be processed, the orchestration services 410 can provide workflow(s) to the orchestration engine 210 and execute a selected workflow, for example. The algorithm orchestrator 210 and associated processing electronics 230 can be located on a local system, on a cloud-based system (e.g., the cloud-based system 100 of FIG. 1, etc.), on an edge device connecting a local system to a cloud-based system, etc.

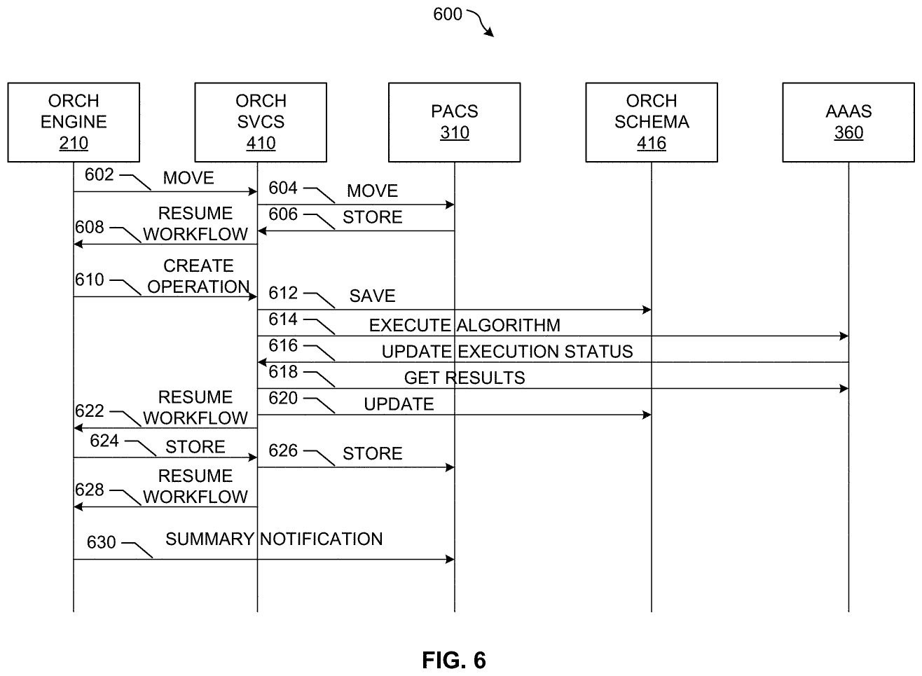

FIG. 6 depicts an example data flow 600 to orchestrate workflow execution using the algorithm orchestrator 210. In the example of FIG. 6, the orchestration engine 210 sends a move command 602 for an image study or other exam to orchestration services 410, which sends a move command 604 for the study/exam to the PACS 310 and/or other data source storing the study/exam. The PACS 310 responds by storing 606 the study/exam with the orchestration services 410. The orchestration services 410 triggers the orchestration engine 410 to resume 608 a selected workflow for the image/study. The orchestration engine 410 then creates an operation 610 for the orchestration services 410 to apply to the algorithm to the image/study. The orchestration services 410 saves 612 information with the orchestration schema database 416.

The orchestration services 410 also triggers execution of an algorithm 314 at the AAAS 360. The AAAS 360 updates an execution status 616 of the algorithm with respect to the study/exam data for the orchestration services 410. The orchestration services 410 gets results 618 from the AAAS 360 once algorithm execution is complete. The orchestration services 410 updates the orchestration schema 416 based on results of the algorithm execution. The orchestration services 410 also triggers the orchestrator 210 to resume the workflow, and the algorithm orchestrator 210 triggers the orchestration services 410 to store results of the algorithm execution, and the orchestration services 410 stores 626 the information at the PACS 310. The orchestration services 410 then tells the orchestrator 210 to resume the workflow 628. The orchestration engine 210 provides a summary notification 630 to the PACS 310.

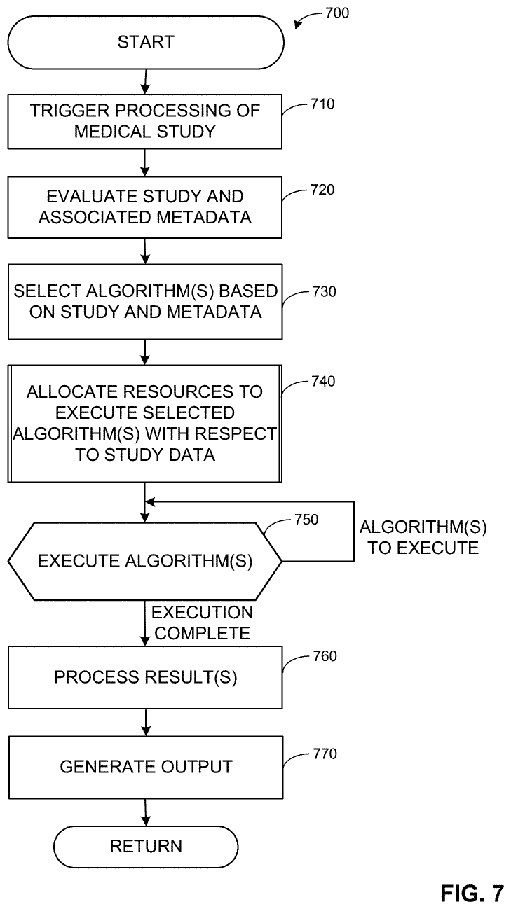

FIG. 7 illustrates a flow diagram of an example method 700 to process a medical study (e.g., an exam, an image study, etc.). At block 710, processing of the medical study is triggered. For example, arrival of the study at the information system (e.g., PACS, etc.) 310, RIS 320, and/or other DICOM source 240 can trigger processing of the study by the algorithm orchestrator 210 and orchestration services 410. Selection of the study from a worklist via the viewer 330 can trigger processing of the study, for example.

At block 720, the study and associated metadata are evaluated to determine one or more criterion for selection of algorithm(s) to apply to the study data. For example, the study and associated metadata are processed by the orchestrator 210 and associated services 410 to identify the type of study, associated modality, anatomy(-ies) of interest, etc. At block 730, one or more algorithms are selected based on the evaluation of the study and associated metadata. For example, presence of a lung image and an indication of shortness of breath in the image metadata can trigger selection via the AAAS 360 of a pneumothorax detection algorithm to process the study data to determine the presence or likely presence of a pneumothorax.

At block 740, resources are allocated to execute the selected algorithm(s) to process the study data. For example, one or more models 370 (e.g., neural network models, other machine learning, deep learning, and/or other artificial intelligence models, etc.) can be deployed to implement one or more selected algorithms. For example, a neural network model can be used to implement an ET tube detection algorithm, pneumothorax detection algorithm, lung segmentation algorithm, node detection algorithm, etc. In certain examples, the model(s) 370 can be trained and/or deployed using the inferencing engine 380 based on ground truth and/or other verified data to develop nodes, interconnections between nodes, and weights on nodes/connections, etc., to implement an algorithm using the model 370. The algorithm can then be applied to study data by passing the data into the model 370 and capturing the model output, for example. Other model(s) can be developed and provided for algorithm implementation based on modality, anatomy, protocol, condition, etc., using the AAAS 360, orchestrator schema 416, AAAS database 420, etc.

At block 750, the selected algorithm(s) are executed with respect to the medical study data. For example, the medical study data is fed into and/or otherwise input to the model(s) 370, inferencing engine 380, other analytics provided by the AAAS 360, etc., to generate one or more results from algorithm execution. For example, the pneumothorax model processes medical study lung image data to determine whether or not a pneumothorax is present in the lung image; an ET tube model processes medical study image data to determine positioning of the ET tube and verify proper placement for the patient; etc.

At block 760, result(s) from the executed algorithm(s) are processed. For example, results from several algorithms can be combined into a determination of patient diagnosis, patient treatment, corrective action (e.g., the ET tube is misplaced and is to be repositioned, a pneumothorax is present and is to be alleviated, etc.). One or more yes/no, positive/negative, present/absent, probability, and/or other outcome from individual model 370 algorithmic processing can be further processed to drive a clinical determination, corrective action, reporting, display, etc.

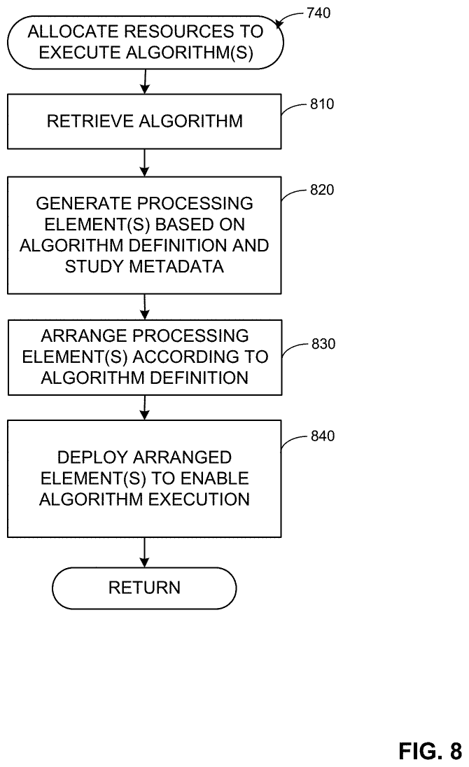

FIG. 8 illustrates an example flow diagram to allocate resources to execute algorithms with respect to medical study data (e.g., block 740 of the example of FIG. 7). At block 810, an algorithm is retrieved (e.g., from the orchestration schema 416, the AAAS database 420, the file share 430, etc.). The algorithm and its definition are retrieved based on its selection for applicability to the medical study data, for example.

At block 820, processing element(s) are generated based on a definition of the algorithm and metadata associated with the study. For example, one or more artificial intelligence (e.g., machine learning, deep learning, etc.) network model constructs 370, one or more virtual machines and/or containers, one or more processors, etc., is allocated and/or instantiated based on the definition of the algorithm and study metadata. At block 830, the processing element(s) are organized according to the algorithm definition. For example, multiple AI models 370 can be arranged in parallel, in series, etc., to implement the algorithm according to its definition, customized to fit the study data to be applied to the algorithm.

At block 840, the arranged processing element(s) is/are deployed to enable execution of the algorithm with respect to the study data. For example, one or more models 370 (e.g., neural network models, other machine learning, deep learning, and/or other artificial intelligence models, etc.) can be deployed to implement one or more selected algorithms. For example, a neural network model can be used to implement an ET tube detection algorithm, pneumothorax detection algorithm, lung segmentation algorithm, node detection algorithm, etc. In certain examples, the model(s) 370 can be trained and/or deployed using the inferencing engine 380 based on ground truth and/or other verified data to develop nodes, interconnections between nodes, and weights on nodes/connections, etc., to implement an algorithm using the model 370. The algorithm can then be applied to study data by passing the data into the model 370 and capturing the model output, for example. Other model(s) 370 can be developed and provided for algorithm implementation based on modality, anatomy, protocol, condition, etc., using the AAAS 360, orchestrator schema 416, AAAS database 420, etc. The algorithm orchestrator 210 leverages the AAAS 360 and the orchestrator services 410 to apply the deployed set of processing element(s) to the study data to obtain result(s) (e.g., at block 760 of the example of FIG. 7), for example.

FIGS. 9-11 illustrate example algorithms dynamically constructed by the algorithm orchestrator 210 from a plurality of node models. For example, FIG. 9 illustrates an algorithm 900 applying a pneumothorax (PTX) model 940 to a DICOM study when the modality is CR or DX 910, the view position is AP or PA 920, and the study description is a chest image series 930. A series of decisions 910, 920, 930 is used to evaluate the study data before applying the model 940 to detect pneumothorax when all of the decisions/conditions are satisfied. The algorithm then ends with a result of yes or no, 1 or 0, present or absent, positive or negative, malignant or benign, etc., in answer to the pneumothorax model analysis.

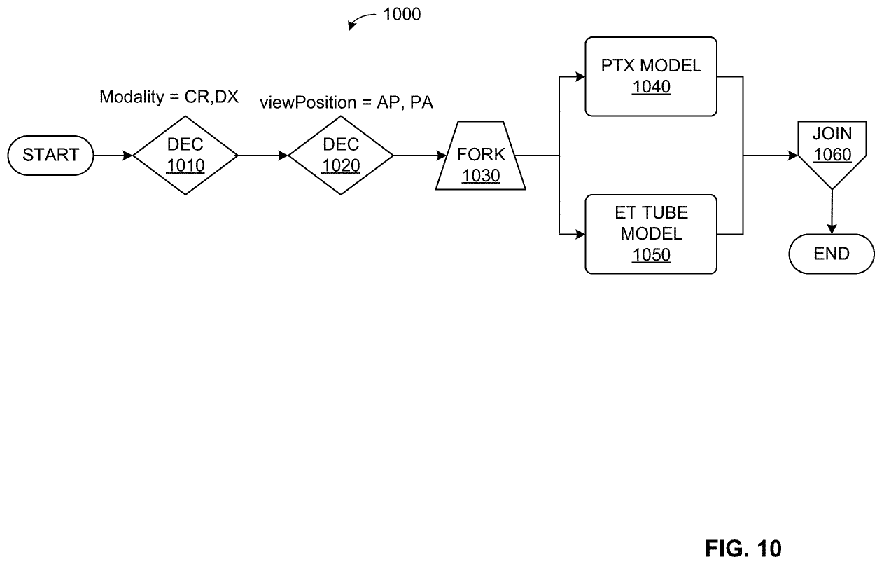

FIG. 10 illustrates another example algorithm 1000 constructed from a plurality of model constructs forming nodes in the algorithm model. In the example of FIG. 10, a series of decisions 1010, 1020 (e.g., is the modality CR or DX 1010 and is the view position AP or PA 1020) results in a fork 1030 to apply multiple models 1040, 1050 to the DICOM study data. In this example, both a PTX model 1040 and an ET tube model 1050 are applied to the DICOM data, and the results are joined 1060 to form a result of the algorithm. Thus, in the example of FIG. 10, both ET tube placement and pneumothorax detection are combined to determine a result indicating whether or not the associated patient has an issue to be addressed.

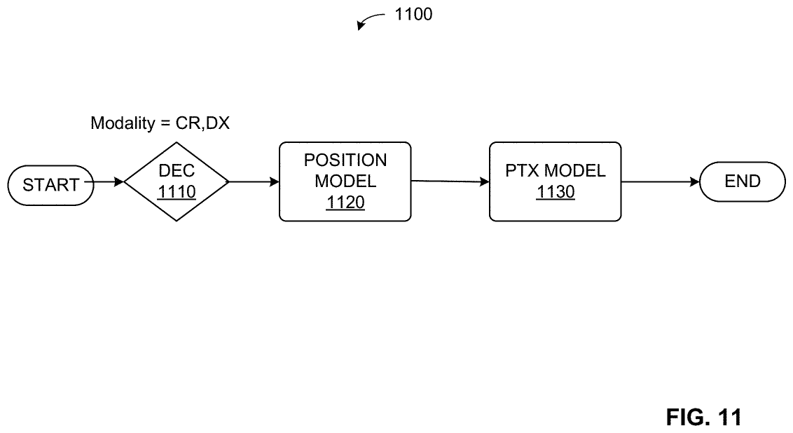

FIG. 11 illustrates another example algorithm 1100 constructed from a plurality of model constructs forming nodes in the algorithm model. In the example of FIG. 11, a decision node 1110 evaluates whether the modality is CR or DX. If so, then a position model 1120 is first applied to the DICOM study data. Then, based on an output of that model 1120, a PTX model 1130 is applied to determine an ultimate result of the algorithm. Thus, FIG. 10 illustrates an example algorithm that applies models in parallel to DICOM study data, and FIG. 11 illustrates an example algorithm that applies models in series to the DICOM study data.

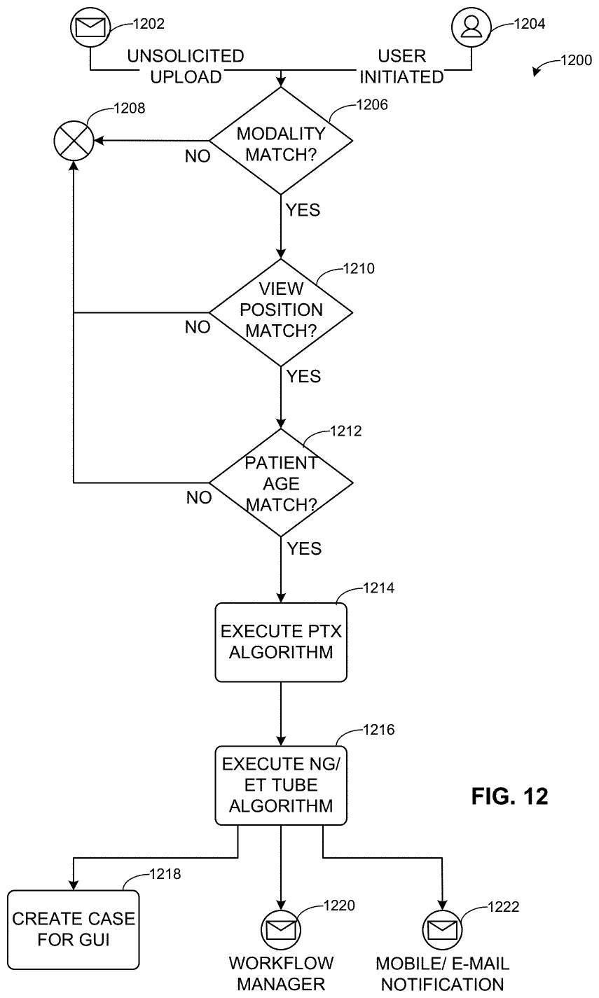

FIG. 12 illustrates a flow diagram of an example algorithm orchestration process 1200 to augment clinical workflows using the algorithm orchestrator 210. As shown in the example of FIG. 12, orchestration can begin with an unsolicited upload of a medical imaging study (block 1202) or initiated by a user with respect to a medical imaging study (block 1204). The study (e.g., DICOM header information and/or other metadata associated with the study) is then evaluated to determine whether the imaging modality matches one or more set criterion (block 1206). If not, then the evaluation ends (block 1208). If the modality matches the criterion(-ia), then the study is evaluated to determine whether the view position matches one or more set criterion (block 1210). If not, then the evaluation ends (block 1208). If the view position matches the criterion(-ia), then the study is evaluated to determine whether the age of the patient associated with the study matches one or more set criterion (block 1212). If not, then the evaluation ends (block 1208). If the patient age matches the criterion(-ia), then a pneumothorax algorithm is executed with respect to the study data (block 1214). A tube positioning algorithm (e.g., ET tube and/or nasogastric (NG) tube placement detection algorithm, etc.) is executed with respect to the study data (block 1216). Output of the models can then be used to create a case for use interaction via a graphical user interface (block 1218) as well as update a workflow manager (block 1220) and practitioner mobile/email notification (block 1222).

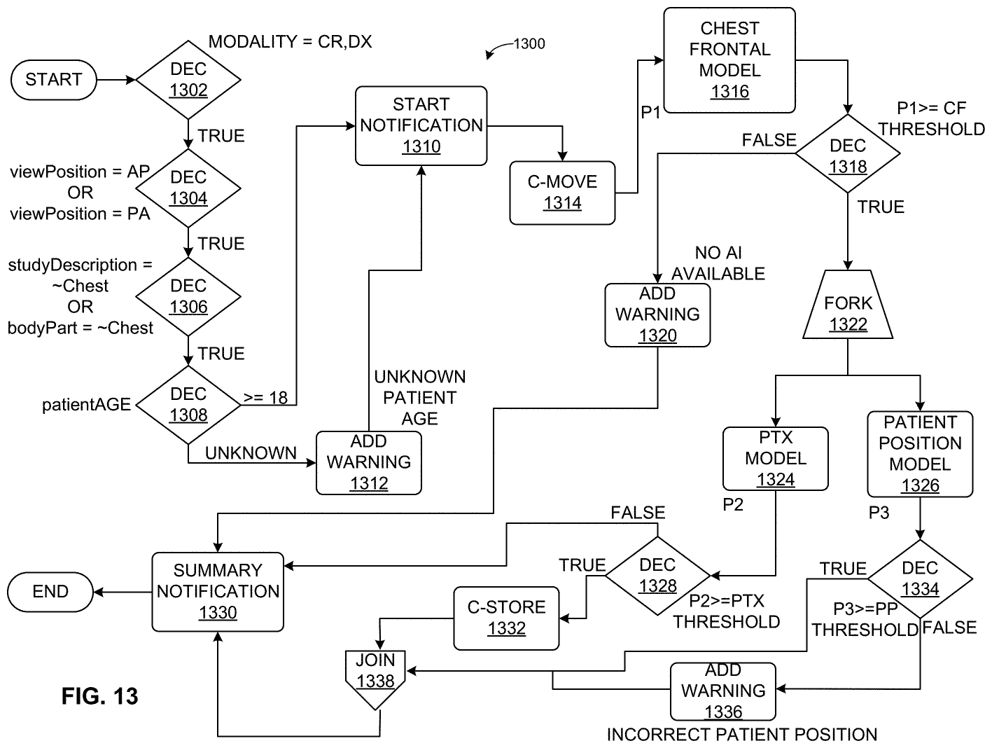

FIG. 13 depicts an example chest x-ray workflow 1300 for pneumothorax (PTX) detection that can be assembled and executed via the algorithm orchestrator 210. The example workflow 1300 is constructed from a plurality of functionality nodes or modules implemented using AI models 370, virtual machines/containers, processors, etc., via the algorithm orchestrator 210, orchestration services 410, AAAS 360, etc. Medical data is processed to determine whether an imaging modality used to obtain the medical data is CR or DX (block 1302). Medical data is processed to determine whether a view position of an image in the medical data is AP or PA (block 1304). Medical data is processed to determine whether the medical study is a chest study or a body part included in the medical data is a chest (block 1306). Patient age is also evaluated in the medical data (block 1308). If the patient is 18 or older, a notification is generated to move the medical data and start analysis (block 1310). However, if the patient is less than 18 years old, then a warning is added (block 1312) to indicate that the patient is a minor and/or the patient's age is unknown, for example.

The medical data is moved for algorithm construction and processing (block 1314) and provided to a chest frontal model for analysis (block 1316). A chest frontal output P1 of the model is evaluated with respect to a chest frontal (CF) threshold (block 1318). If the model output P1 is less than the CF threshold, then a warning is generated indicating that further analytics cannot/will not be applied (block 1320) and a summary notification is generated (block 1330). If the model output P1 is greater than or equal to the CF threshold, then a fork (block 1322) sends medical data into a PTX model (block 1324) and a patient position model (block 1326). An output P2 of the PTX model is evaluated to determine whether the output P2 is greater than or equal to a pneumothorax (PTX) threshold (block 1328). If not, then a summary notification is generated (block 1330). If the model output P2 is greater than or equal to the PTX threshold, then the analysis is stored for further processing (e.g., added to a worklist, routed to another system, etc.) (block 1332). An output P3 of the patient position model is compared to a patient position (PP) threshold (block 1334). When the output P3 is not greater than or equal to the PP threshold, a warning is generated (block 1336). If the output P3 is greater than or equal to the PP threshold, the P3 output and the P2 output are joined (block 1338). The joined output can then be used to generate a summary notification (block 1330) for user interface display via the viewer 330, storage in the file share 340, information system 310, RIS 320, DICOM source 240, schema 414-418, data store 420, etc.

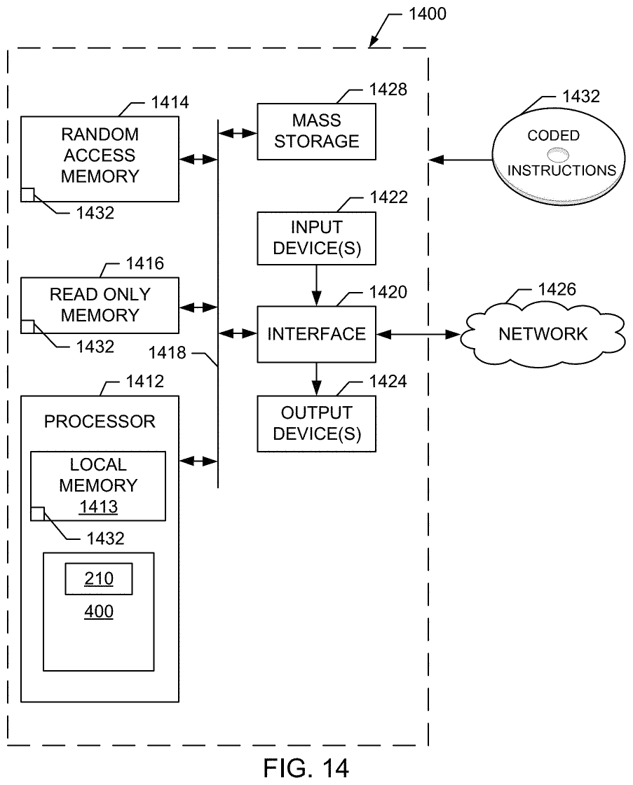

Flowcharts, flow diagrams, and data flows representative of example machine readable instructions for implementing and/or executing in conjunction with the example systems/apparatus of FIGS. 1-4 are shown above in FIGS. 5-13. In these examples, the machine readable instructions comprise a program for execution by a processor such as the processor 1412 shown in the example processor platform 1400 discussed below in connection with FIG. 14. The program can be embodied in software stored on a tangible computer readable storage medium such as a CD-ROM, a floppy disk, a hard drive, a digital versatile disk (DVD), a BLU-RAY™ disk, or a memory associated with the processor 1412, but the entire program and/or parts thereof could alternatively be executed by a device other than the processor 1412 and/or embodied in firmware or dedicated hardware. Further, although the example program is described with reference to the flowchart and/or process(es) illustrated in FIGS. 5-13, many other methods of implementing the examples disclosed and described here can alternatively be used. For example, the order of execution of the blocks can be changed, and/or some of the blocks described can be changed, eliminated, or combined.

As mentioned above, the example process(es) of FIGS. 5-13 can be implemented using coded instructions (e.g., computer and/or machine readable instructions) stored on a tangible computer readable storage medium such as a hard disk drive, a flash memory, a read-only memory (ROM), a compact disk (CD), a digital versatile disk (DVD), a cache, a random-access memory (RAM) and/or any other storage device or storage disk in which information is stored for any duration (e.g., for extended time periods, permanently, for brief instances, for temporarily buffering, and/or for caching of the information). As used herein, the term tangible computer readable storage medium is expressly defined to include any type of computer readable storage device and/or storage disk and to exclude propagating signals and to exclude transmission media. As used herein, “tangible computer readable storage medium” and “tangible machine readable storage medium” are used interchangeably. Additionally or alternatively, the example process(es) of FIGS. 5-13 can be implemented using coded instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media. As used herein, when the phrase “at least” is used as the transition term in a preamble of a claim, it is open-ended in the same manner as the term “comprising” is open ended.

The subject matter of this description may be implemented as stand-alone system or for execution as an application capable of execution by one or more computing devices. The application (e.g., webpage, downloadable applet or other mobile executable) can generate the various displays or graphic/visual representations described herein as graphic user interfaces (GUIs) or other visual illustrations, which may be generated as webpages or the like, in a manner to facilitate interfacing (receiving input/instructions, generating graphic illustrations) with users via the computing device(s).

Memory and processor as referred to herein can be stand-alone or integrally constructed as part of various programmable devices, including for example a desktop computer or laptop computer hard-drive, field-programmable gate arrays (FPGAs), application-specific integrated circuits (ASICs), application-specific standard products (ASSPs), system-on-a-chip systems (SOCs), programmable logic devices (PLDs), etc. or the like or as part of a Computing Device, and any combination thereof operable to execute the instructions associated with implementing the method of the subject matter described herein.

Computing device as referenced herein can include: a mobile telephone; a computer such as a desktop or laptop type; a Personal Digital Assistant (PDA) or mobile phone; a notebook, tablet or other mobile computing device; or the like and any combination thereof.

Computer readable storage medium or computer program product as referenced herein is tangible (and alternatively as non-transitory, defined above) and can include volatile and non-volatile, removable and non-removable media for storage of electronic-formatted information such as computer readable program instructions or modules of instructions, data, etc. that may be stand-alone or as part of a computing device. Examples of computer readable storage medium or computer program products can include, but are not limited to, RAM, ROM, EEPROM, Flash memory, CD-ROM, DVD-ROM or other optical storage, magnetic cassettes, magnetic tape, magnetic disk storage or other magnetic storage devices, or any other medium which can be used to store the desired electronic format of information and which can be accessed by the processor or at least a portion of the computing device.

The terms module and component as referenced herein generally represent program code or instructions that causes specified tasks when executed on a processor. The program code can be stored in one or more computer readable mediums.

Network as referenced herein can include, but is not limited to, a wide area network (WAN); a local area network (LAN); the Internet; wired or wireless (e.g., optical, Bluetooth, radio frequency (RF)) network; a cloud-based computing infrastructure of computers, routers, servers, gateways, etc.; or any combination thereof associated therewith that allows the system or portion thereof to communicate with one or more computing devices.

The term user and/or the plural form of this term is used to generally refer to those persons capable of accessing, using, or benefiting from the present disclosure.

FIG. 14 is a block diagram of an example processor platform 1400 capable of executing instructions to implement the example systems and methods disclosed and described herein. The processor platform 1400 can be, for example, a server, a personal computer, a mobile device (e.g., a cell phone, a smart phone, a tablet such as an IPAD™), a personal digital assistant (PDA), an Internet appliance, or any other type of computing device.

The processor platform 1400 of the illustrated example includes a processor 1412. The processor 1412 of the illustrated example is hardware. For example, the processor 1412 can be implemented by one or more integrated circuits, logic circuits, microprocessors or controllers from any desired family or manufacturer.

The processor 1412 of the illustrated example includes a local memory 1413 (e.g., a cache). The processor 1412 of the illustrated example is in communication with a main memory including a volatile memory 1414 and a non-volatile memory 1416 via a bus 1418. The volatile memory 1414 can be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 1416 can be implemented by flash memory and/or any other desired type of memory device. Access to the main memory 1414, 1416 is controlled by a memory controller.

The processor platform 1400 of the illustrated example also includes an interface circuit 1420. The interface circuit 1420 can be implemented by any type of interface standard, such as an Ethernet interface, a universal serial bus (USB), and/or a PCI express interface.

In the illustrated example, one or more input devices 1422 are connected to the interface circuit 1420. The input device(s) 1422 permit(s) a user to enter data and commands into the processor 1412. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 1424 are also connected to the interface circuit 1420 of the illustrated example. The output devices 1424 can be implemented, for example, by display devices (e.g., a light emitting diode (LED), an organic light emitting diode (OLED), a liquid crystal display, a cathode ray tube display (CRT), a touchscreen, a tactile output device, a light emitting diode (LED), a printer and/or speakers). The interface circuit 1420 of the illustrated example, thus, typically includes a graphics driver card, a graphics driver chip or a graphics driver processor.

The interface circuit 1420 of the illustrated example also includes a communication device such as a transmitter, a receiver, a transceiver, a modem and/or network interface card to facilitate exchange of data with external machines (e.g., computing devices of any kind) via a network 1426 (e.g., an Ethernet connection, a digital subscriber line (DSL), a telephone line, coaxial cable, a cellular telephone system, etc.).

The processor platform 1400 of the illustrated example also includes one or more mass storage devices 1428 for storing software and/or data. Examples of such mass storage devices 1428 include floppy disk drives, hard drive disks, compact disk drives, Blu-ray disk drives, RAID systems, and digital versatile disk (DVD) drives.

The coded instructions 1432 can be stored in the mass storage device 1428, in the volatile memory 1414, in the non-volatile memory 1416, and/or on a removable tangible computer readable storage medium such as a CD or DVD. The instructions 1432 can be executed by the processor 1412 to implement the example system(s) 100-400, etc., as disclosed and described above.

From the foregoing, it will be appreciated that example methods, apparatus and articles of manufacture have been disclosed that provide dynamic, study-specific generation of algorithms and processing resources for medical data. The disclosed methods, apparatus and articles of manufacture improve the efficiency of using a computing device and an interface being driven by the computing device to accept a study, evaluate the study and its metadata, and then dynamically select and/or generate algorithm(s) and associated processing elements constructed for that study to process the study and drive an actionable result. Certain examples improve a computer system and its processing and interoperability through connection with a cloud and/or edge device and services that can be dynamically allocated and customized for particular data, diagnostic criteria, treatment goals, etc. in a manner previously unavailable. Certain examples alter the operation of the computing device and provide a new interface and interaction to dynamically instantiate algorithms using processing elements to process medical study data. The disclosed methods, apparatus and articles of manufacture are accordingly directed to one or more improvement(s) in the functioning of a computer, as well as a new medical data processing methodology and infrastructure.

Thus, rather than static image and/or other medical data processing algorithms, certain examples enable dynamic algorithm matching and workflow generation to specific patient exams and/or image studies. Certain examples dynamically match an exam/study to one or more algorithms based on exam/study type (e.g., reason for exam, modality, clinical focus, etc.), exam/study content (e.g., included anatomy, reason for exam, etc.), etc. As such, exam/study data can be routed to one or more dynamically instantiated processing models to apply one or more algorithms to the data to obtain a result (e.g., a segmented image, computer-aided detection and/or diagnosis of objects in an image, object labeling in an image, feature identification in an image, region of interest identification in an image, change in a series of images, other processed image, etc.) and drive further action by a system such as triggering follow-up in a RIS, PACS, EMR, laboratory testing system, scheduler, follow-up image acquisition, etc.

Certain examples can operate on a complete medical study, on partial medical data streamed, etc. Certain examples analyze anatomy, modality, reason for exam, etc., to allocate processing elements to implement algorithms to process medical data accordingly. Certain examples detect anatomy in the medical data, form feature vectors from the medical data, etc., to identify and characterize the medical data for corresponding customized algorithm generation and application. As a result, actions triggered by algorithm execution can include analysis generated in a graphical user interface display, further action triggered in a health system, prioritization of the study in a worklist, notification to a clinician and/or system of results, update of the original medical study with results, etc.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. An apparatus comprising:
an algorithm orchestrator to analyze medical data and associated metadata and select an algorithm based on the analysis; and
a postprocessor to execute the algorithm with respect to the medical data using one or more processing elements, the one or more processing elements dynamically selected and arranged in combination by the algorithm orchestrator to implement the algorithm for the medical data, the postprocessor to output a result of the algorithm for action by the algorithm orchestrator.

2. The apparatus of claim 1, wherein the one or more processing elements include one or more artificial intelligence models to instantiate the algorithm.

3. The apparatus of claim 1, wherein the postprocessor includes at least one of an inferencing engine and services to form and arrange the one or more processing elements into the algorithm as triggered by the algorithm orchestrator.

4. The apparatus of claim 1, wherein the postprocessor is to store the arranged combination of one or more processing elements for later retrieval to implement the algorithm.

5. The apparatus of claim 1, wherein the arrangement of one or more processing elements includes a fork to apply a plurality of processing elements in parallel to the medical data.

6. The apparatus of claim 1, wherein the arrangement of one or more processing elements includes a join to combine output of a plurality of processing elements into a result.

7. The apparatus of claim 1, wherein the algorithm orchestrator is to select a plurality of algorithms combined by the algorithm orchestrator and instantiated by the postprocessor to apply a plurality of algorithms to the medical data.

8. The apparatus of claim 1, wherein the action includes at least one of: a) displaying, via a viewer a graphical user interface generated with the algorithm result; or b) trigger a health system to process the medical data based on the result.

9. A computer-readable storage medium including instructions which, when executed by at least one processor, cause the at least one processor to at least:
analyze medical data and associated metadata of a medical study;
select an algorithm based on the analysis; and
dynamically select, arrange, and configure processing elements in combination to implement the algorithm for the medical data;
execute the algorithm with respect to the medical data using the arranged, configured processing elements; and
output an actionable result of the algorithm for the medical study.

10. The computer-readable storage medium of claim 9, wherein the processing elements include one or more artificial intelligence models to instantiate the algorithm.

11. The computer-readable storage medium of claim 9, wherein the instructions, when executed, cause the at least one processor to store the arranged combination of processing elements for later retrieval to implement the algorithm.

12. The computer-readable storage medium of claim 9, wherein the arrangement of processing elements includes a fork to apply at least a portion of the plurality of processing elements in parallel to the medical data.

13. The computer-readable storage medium of claim 9, wherein the arrangement of processing elements includes a join to combine output of the plurality of processing elements into a result.

14. The computer-readable storage medium of claim 9, wherein the instructions, when executed, cause the at least one processor to select a plurality of algorithms combined and instantiated to apply a plurality of algorithms to the medical data.

15. The computer-readable storage medium of claim 9, wherein the instructions, when executed, cause the at least one processor to trigger at least one of: a) displaying, via a viewer a graphical user interface generated with the algorithm result; or b) trigger a health system to process the medical data based on the result.

16. A computer-implemented method comprising:
analyzing, by executing an instruction with at least one processor, medical data and associated metadata of a medical study;
selecting, by executing an instruction with the at least one processor, an algorithm based on the analysis; and
dynamically selecting, arranging, and configuring, by executing an instruction with the at least one processor, processing elements in combination to implement the algorithm for the medical data;
executing, by executing an instruction with the at least one processor, the algorithm with respect to the medical data using the arranged, configured processing elements; and
outputting, by executing an instruction with the at least one processor, an actionable result of the algorithm for the medical study.

17. The method of claim 16, wherein the processing elements include one or more artificial intelligence models to instantiate the algorithm.

18. The method of claim 16, father including storing the arranged combination of processing elements for later retrieval to implement the algorithm.

19. The method of claim 16, further including selectin a plurality of algorithms combined and instantiated to apply a plurality of algorithms to the medical data.

20. The method of claim 16, further including triggering at least one of: a) displaying, via a viewer a graphical user interface generated with the algorithm result; or b) trigger a health system to process the medical data based on the result.
